(19) United States
(12) Patent Application Publication
(10) Pub. No.: US 2020/0174119 A1
(43) Pub. Date: Jun. 4, 2020

(54) ULTRASOUND IMAGING SYSTEM AND METHOD FOR MEASURING A VOLUME FLOW RATE

(71) Applicant: General Electric Company, Schenectady, NY (US)

(72) Inventors: Rimon Tadross, Milwaukee, WI (US); David Dubberstein, Waukesha, WI (US)

(21) Appl. No.: 16/209,775

(22) Filed: Dec. 4, 2018

(51) Int. Cl. G01S 15/89; G01S 7/52; A61B 8/06; A61B 8/00

(52) U.S. Cl. CPC G01S 15/8979; G01S 7/5208; A61B 8/467; A61B 8/06; G01S 7/52071

(57) ABSTRACT

An ultrasound imaging system and method includes acquiring and displaying a first image of a first plane including a longitudinal axis of a vessel and identifying first position information of the longitudinal axis. The system and method includes acquiring and displaying a second image of a second plane that intersects the longitudinal axis of the vessel at an oblique angle, where the second plane is rotated about the longitudinal axis of the ultrasound probe, where the ultrasound probe is in the same position with respect to the vessel when acquiring both the first image of the first plane and the second image of the second plane, and identifying second position information defining the second plane with respect to the ultrasound probe. The system and method include calculating a volume flow rate based on the first image, the second image, the first position information and the second position information.

### CROSS-REFERENCE TO RELATED APPLICATIONS/INCORPORATION BY REFERENCE

This application makes reference to:

U.S. application Ser. No. 16/209,755 (Attorney Docket No. 325815-US-1), filed on even date herewith.

The above referenced application is hereby incorporated herein by reference in its entirety.

### BACKGROUND OF THE INVENTION

This invention relates generally to ultrasound imaging and, more particularly, to a method and ultrasound imaging system for measuring a volume flow rate through a vessel.

Ultrasound Doppler imaging is commonly used to detect the presence of blood flow in the body. Flow velocities at a given location in the vessel can be estimated using the measured Doppler shift and correcting for the Doppler angle between the ultrasound beams and the vessel orientation. Even so, the calculation of volume flow cannot be performed without making assumptions regarding the vessel geometry and the flow profile within the vessel when using conventional techniques. The most common method for estimating volume flow rate is performed by multiplying the mean spatial velocity imaged within the vessel by the vessel cross-sectional area. In this method, the vessel cross-sectional area is estimated by assuming a circular vessel cross-section and flow velocity is determined by pulse wave Doppler. Pulse wave Doppler calculates the Doppler shift of ultrasound signals within a Doppler gate and uses the Doppler shift to estimate the velocity. Pulse wave Doppler only estimates the velocity within the Doppler gate. Assuming that the vessel cross-section is circular and assuming that the flow in the entire vessel is the same as the region within the Doppler gate introduces significant error into conventional volume flow rate calculations. As a result of the potential for error, many clinicians either do not use or do not rely on volume flow rates provided by conventional ultrasound techniques

Therefore, for at least the reasons discussed above, a need exists for an improved method and ultrasound imaging system for calculating volume flow rate. Additionally, it would be beneficial if the improved method and system for calculating volume flow rate would provide volume flow rates in real-time.

### BRIEF DESCRIPTION OF THE INVENTION

In one embodiment, a method for calculating a volume flow rate using ultrasound includes acquiring, with an ultrasound probe, a first image of a first plane, where the first plane includes a longitudinal axis of a vessel. The method includes displaying the first image on a display device. The method includes identifying, with a processor, first position information, where the first position information is of the longitudinal axis with respect to the ultrasound probe. The method includes acquiring, with the ultrasound probe, a second image of a second plane that intersects the longitudinal axis of the vessel at an oblique angle, where the second plane is rotated about a longitudinal axis of the ultrasound probe with respect to the first plane, and where the ultrasound probe is in the same position with respect to the vessel when acquiring both the first image of the first plane and the second image of the second plane. The method includes displaying the second image on the display device. The method includes identifying, with the processor, second position information, where the second position information defines the second plane with respect to the ultrasound probe. The method includes calculating, with the processor, a volume flow rate of the vessel based on the first image, the second image, the first position information, and the second position information, and displaying the volume flow rate on a display device.

In another embodiment, an ultrasound imaging system includes an ultrasound probe comprising a plurality of elements, a display device, and a processor in electronic communication with the ultrasound probe and the display device. The processor is configured to control the ultrasound probe to acquire a first image of a first plane, wherein the first plane is positioned to include a longitudinal axis of a vessel. The processor is configured to display the first image on the display device and identify first position information of the longitudinal axis of the vessel with respect to the ultrasound probe. The processor is configured to control the ultrasound probe to acquire a second image of a second plane, wherein the second plane is rotated about a longitudinal axis of the ultrasound probe from the first plane, and wherein the ultrasound probe is in the same position with respect to the vessel when acquiring both the first image of the first plane and the second image of the second plane. The processor is configured to display the second image on the display device, identify second position information, and calculate a volume flow rate of the vessel based on the first image, the second image, the first position information, and the second position information, and display the volume flow rate on the display device.

### BRIEF DESCRIPTION OF THE DRAWINGS

FIG. 1 is a block diagram of an ultrasound imaging system in accordance with an embodiment;

FIG. 2 is a perspective view of an E4D probe in accordance with an embodiment;

FIG. 3 is a perspective view of a rotating mechanical probe in accordance with an embodiment;

FIG. 4 is a flow chart of a method in accordance with an embodiment;

FIG. 5 is a schematic representation of a vessel, an ultrasound probe, and two planes in accordance with an embodiment;

FIG. 6 is a schematic representation of an image in accordance with an embodiment;

FIG. 7 is a schematic representation of a screenshot in accordance with an embodiment;

FIG. 8 is a schematic representation of a plane with respect to a vessel in accordance with an embodiment;

FIG. 9 is a schematic representation of an image in accordance with an embodiment;

FIG. 10 is a schematic representation of a screenshot in accordance with an embodiment;

FIG. 11 is a schematic representation of a first plane, a second plane, and a third plane with respect to a vessel in accordance with an embodiment;

FIG. 12 is a flow chart of a method in accordance with an embodiment; and

FIG. 13 is a schematic representation of an image in accordance with an embodiment.

### DETAILED DESCRIPTION OF THE INVENTION

The foregoing summary, as well as the following detailed description of certain embodiments of the present invention, will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks may be implemented in a single piece of hardware (e.g., a general purpose signal processor or a block or random access memory, hard disk, or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

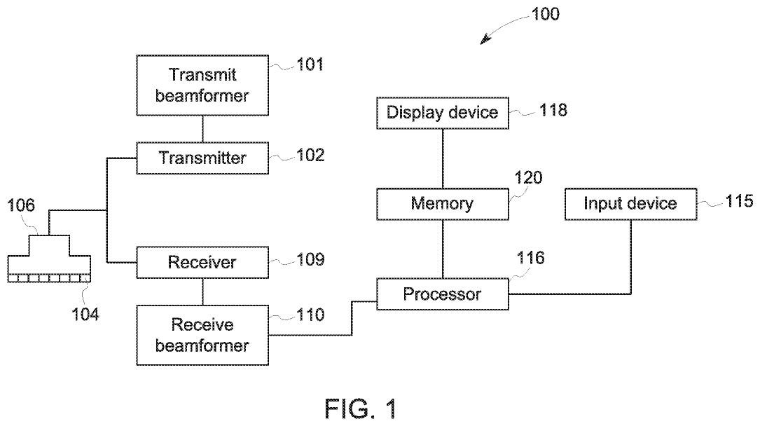

FIG. 1 is a schematic diagram of an ultrasound imaging system 100. The ultrasound imaging system 100 includes a transmit beamformer 101 and a transmitter 102 that drive elements 104 within an ultrasound probe 106 to emit pulsed ultrasonic signals into a patient (not shown). The ultrasound probe 106 may, for instance, be an E4D probe or a mechanically rotating probe. The E4D probe may be a linear E4D probe, a curvilinear E4D probe, or a sector E4D probe. The mechanically rotating probe may be a linear mechanically rotating probe, a curvilinear mechanically rotating probe, or a sector mechanically rotating probe. Additional details about both the E4D probe and the mechanically rotating probe will be discussed hereinafter. The ultrasound probe 106 may be configured to acquire both 2D B-mode data and 2D colorflow data or both 2D B-mode data and another ultrasound mode that detects blood flow velocity in the direction of a vessel axis. The ultrasound probe 106 may have the elements 104 arranged in a 1D array or in a 2D array. Still referring to FIG. 1, the pulsed ultrasonic signals are back-scattered from structures in the body, like blood cells or muscular tissue, to produce echoes that return to the elements 104. The echoes are converted into electrical signals, or ultrasound data, by the elements 104, and the electrical signals are received by a receiver 109. The electrical signals representing the received echoes are passed through a receive beamformer 110 that outputs ultrasound data. According to some embodiments, the ultrasound probe 106 may contain electronic circuitry to do all or part of the transmit beamforming and/or the receive beamforming. For example, all or part of the transmit beamformer 101, the transmitter 102, the receiver 109, and the receive beamformer 110 may be situated within the ultrasound probe 106. The terms “scan” or “scanning” may also be used in this disclosure to refer to acquiring data through the process of transmitting and receiving ultrasonic signals. The terms “data” and “ultrasound data” may be used in this disclosure to refer to either one or more datasets acquired with an ultrasound imaging system. The ultrasound imaging system 100 includes an input device 115. The input device 115 may be used to control the input of patient data or to select various modes, operations, parameters, and the like. The input device 115 may include one or more of a keyboard, a dedicated hard key, a touch pad, a mouse, a track ball, a rotary control, a slider, and the like. The input device 115 may include a proximity sensor configured to detect objects or gestures that are within several centimeters of the proximity sensor. The proximity sensor may be located on either the display device 118 or as part of a touch screen. The input device 115 may include a touch screen that is positioned in front of the display device 118 or the touch screen may be separate from the display device 118. The input device 115 may also include one or more physical controls (such as buttons, sliders, rotary knobs, keyboards, mice, trackballs, etc.) either alone or in combination with graphical user interface icons displayed on the display screen. According to some embodiments, the input device 115 may include a combination of physical controls (such as buttons, sliders, rotary knobs, keyboards, mice, trackballs, etc.) and user interface icons displayed on either the display device 118 or on a touch-sensitive display screen. The display device 118 may be configured to display a graphical user interface (GUI) from instructions stored in a memory 120. The GUI may include user interface icons to represent commands and instructions. The user interface icons of the GUI are configured so that a user may select commands associated with each specific user interface icon in order to initiate various functions controlled by the GUI. For example, GUI icons may be used to represent windows, menus, buttons, cursors, scroll bars, etc. According to embodiments where the input device 115 includes a touch screen, the touch screen may be configured to interact with the GUI displayed on the display device 118. The touch screen may be a single-touch touch screen that is configured to detect a single contact point at a time or the touch screen may be a multi-touch touch screen that is configured to detect multiple points of contact at a time. For embodiments where the touch screen is a multi-point touch screen, the touch screen may be configured to detect multi-touch gestures involving contact from two or more of a user's fingers at a time. The touch screen may be a resistive touch screen, a capacitive touch screen, or any other type of touch screen that is configured to receive inputs from a stylus or one or more of a user's fingers. According to other embodiments, the touch screen may be an optical touch screen that uses technology such as infrared light or other frequencies of light to detect one or more points of contact initiated by a user.

According to various embodiments, the input device 115 may include an off-the-shelf consumer electronic device such as a smartphone, a tablet, a laptop, etc. For purposes of this disclosure, the term “off-the-shelf consumer electronic device” is defined to be an electronic device that was designed and developed for general consumer use and not specifically designed for use in a medical environment. According to some embodiments, the consumer electronic device may be physically separate from the rest of the ultrasound imaging system. The consumer electronic device may communicate with a processor 116 through a wireless protocol, such Wi-Fi, Bluetooth, Wireless Local Area Network (WLAN), near-field communication, etc. According to an embodiment, the consumer electronic device may communicate with the processor 116 through an open Application Programming Interface (API).

The ultrasound imaging system 100 also includes the processor 116 to control the transmit beamformer 101, the transmitter 102, the receiver 109, and the receive beamformer 110. The processor 116 is configured to receive inputs from the input device 115. The receive beamformer 110 may be either a conventional hardware beamformer or a software beamformer according to various embodiments. If the receive beamformer 110 is a software beamformer, it may comprise one or more of the following components: a graphics processing unit (GPU), a microprocessor, a central processing unit (CPU), a digital signal processor (DSP), or any other type of processor capable of performing logical operations. The receive beamformer 110 may be configured to perform conventional beamforming techniques as well as techniques such as retrospective transmit beamforming (RTB). If the receive beamformer 110 is a software beamformer, the processor 116 may be configured to perform some or all of the functions associated with the receive beamformer 110.

The processor 116 is in electronic communication with the ultrasound probe 106. The processor 116 may control the ultrasound probe 106 to acquire ultrasound data. The processor 116 controls which of the elements 104 are active and the shape of a beam emitted from the ultrasound probe 106. The processor 116 is also in electronic communication with the display device 118, and the processor 116 may process the ultrasound data into images for display on the display device 118. The processor 116 may be configured to display one or more non-image elements on the display device 118. The instructions for displaying each of the one or more non-image elements may be stored in the memory 120. For purposes of this disclosure, the term “electronic communication” may be defined to include both wired and wireless connections. The processor 116 may include a central processing unit (CPU) according to an embodiment. According to other embodiments, the processor 116 may include other electronic components capable of carrying out processing functions, such as a digital signal processor, a field-programmable gate array (FPGA), a graphics processing unit (GPU), or any other type of processor. According to other embodiments, the processor 116 may include multiple electronic components capable of carrying out processing functions. For example, the processor 116 may include two or more electronic components selected from a list of electronic components including: a central processing unit (CPU), a digital signal processor (DSP), a field-programmable gate array (FPGA), and a graphics processing unit (GPU). According to another embodiment, the processor 116 may also include a complex demodulator (not shown) that demodulates the RF data and generates raw data. In another embodiment the demodulation may be carried out earlier in the processing chain. The processor 116 may be adapted to perform one or more processing operations according to a plurality of selectable ultrasound modalities on the data. The data may be processed in real-time during a scanning session as the echo signals are received. For the purposes of this disclosure, the term “real-time” is defined to include a procedure that is performed without any intentional delay. Real-time frame rates may vary based on the specific parameters used during the acquisition. The data may be stored temporarily in a buffer during a scanning session and processed in less than real-time. Some embodiments of the invention may include multiple processors (not shown) to handle the processing tasks. For example, an embodiment may use a first processor to demodulate and decimate the RF signal and a second processor to further process the data prior to displaying an image. It should be appreciated that other embodiments may use a different arrangement of processors. For embodiments where the receive beamformer 110 is a software beamformer, the processing functions attributed to the processor 116 and the software beamformer hereinabove may be performed by a single processor, such as the receive beamformer 110 or the processor 116. Or the processing functions attributed to the processor 116 and the software beamformer may be allocated in a different manner between any number of separate processing components.

According to an embodiment, the ultrasound imaging system 100 may continuously acquire real-time ultrasound data at a frame-rate of, for example, 10 Hz to 30 Hz. A live, or real-time, image may be generated based on the real-time ultrasound data. Other embodiments may acquire data and or display the live image at different frame-rates. For example, some embodiments may acquire real-time ultrasound data at a frame-rate of less than 10 Hz or greater than 30 Hz depending on the size of the ultrasound data and the intended application. Other embodiments may use ultrasound data that is not real-time ultrasound data. The memory 120 is included for storing processed frames of acquired data and instructions for displaying one or more non-image elements on the display device 118. In an exemplary embodiment, the memory 120 is of sufficient capacity to store image frames of ultrasound data acquired over a period of time at least several seconds in length. The memory 120 may comprise any known data storage medium. The memory 120 may be a component of the ultrasound imaging system 100, or the memory 120 may be external to the ultrasound imaging system 100 according to other embodiments.

Optionally, embodiments of the present invention may be implemented utilizing contrast agents and contrast imaging. Contrast imaging generates enhanced images of anatomical structures and blood flow in a body when using ultrasound contrast agents including microbubbles. After acquiring data while using a contrast agent, the image analysis includes separating harmonic and linear components, enhancing the harmonic component, and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, data may be processed by other or different mode-related modules by the processor 116 (e.g., B-mode, Color Doppler, M-mode, Color M-mode, spectral Doppler, Elastography, TVI, strain, strain rate and combinations thereof, and the like) to form images or data. For example, one or more modules may generate B-mode, color Doppler, M-mode, color M-mode, spectral Doppler, Elastography, TVI, strain, strain rate and combinations thereof, and the like. The image beams and/or frames are stored and timing information indicating a time at which the data was acquired in memory may be recorded. The modules may include, for example, a scan conversion module to perform scan conversion operations to convert the image frames from beam space coordinates to display space coordinates. A video processor module may be provided that reads the image frames from a memory and displays the image frames in real time while a procedure is being carried out on a patient. A video processor module may store the image frames in an image memory, from which the images are read and displayed.

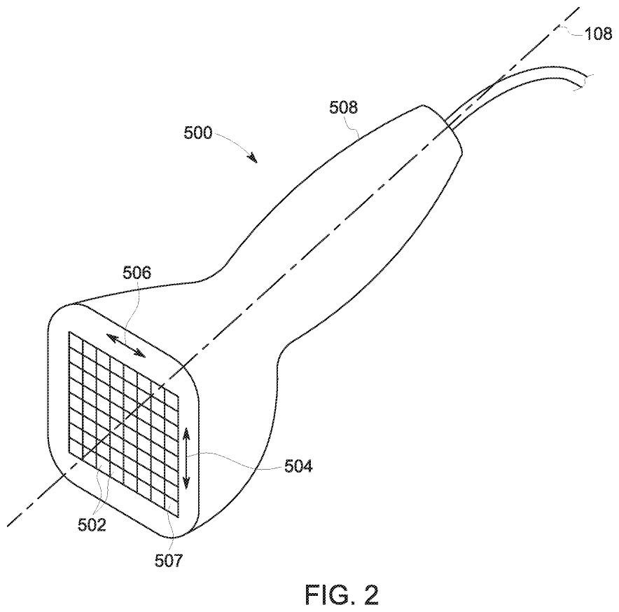

As mentioned previously, the ultrasound probe 106 may be an E4D probe 500 according to an embodiment. FIG. 2 is a perspective view of an E4D probe 500 in accordance with an embodiment. The E4D probe 500 includes a plurality of transducer elements 502 arranged in a 2D matrix array 507. The E4D probe 500 allows for full beamsteering in both an elevation direction 504 and an azimuth 506 direction. This allows the E4D probe 500 to acquire data from any arbitrary plane within a field-of-view of the E4D probe 500 without moving the E4D probe 500 with respect to the anatomical structure being imaged. For example, the longitudinal axis 108 of the ultrasound probe 106 may remain in a fixed position with respect to the anatomical structures, such as the vessel being imaged. The longitudinal axis 108 of the probe 106 is shown with respect to the E4D probe 500. The longitudinal axis 108 is parallel to a long axis of a handle 508 and positioned in the center of the handle. The longitudinal axis 108 intersects the center of the 2D matrix array 507. E4D probes such as the E4D probe 500 are well-known by those skilled in the art in the ultrasound field and will therefore not be described in additional detail.

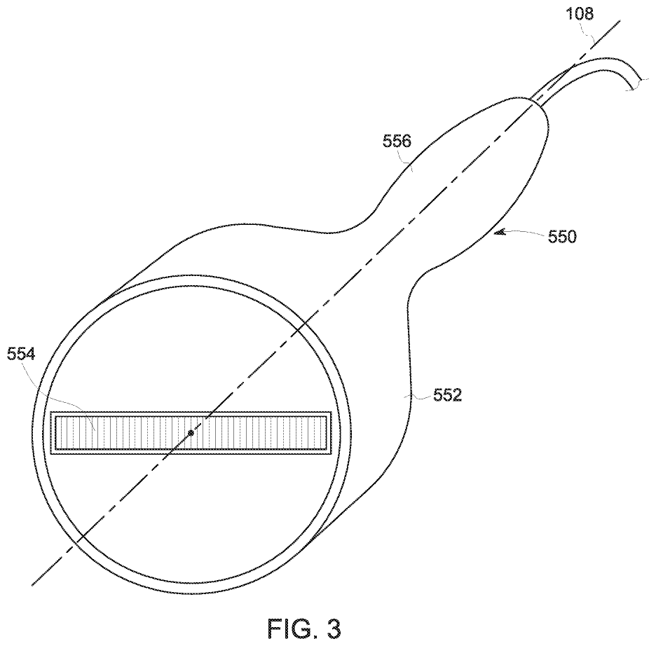

FIG. 3 is a perspective view of a mechanically rotating probe 550 according to an exemplary embodiment. The mechanically rotating probe 550 includes a housing 552. The mechanically rotating probe 550 includes a transducer array 554 that is configured to be rotatable about the longitudinal axis 108 of the ultrasound probe 550. The longitudinal axis 108 is parallel to a long axis of a handle 556 and positioned in the center of the handle 556. The longitudinal axis 108 intersects the center of a transducer array 554. The transducer array 554 may be either a 1D array or a 2D array according to various embodiments. The transducer array 554 may be configured to perform in-plane beamsteering. According to an embodiment, the mechanically rotating probe 550 may include an electric motor or actuator that is configured to cause the transducer array 554 to rotate about the longitudinal axis 108 in response to control signals from the processor 116. The mechanically rotating probe 550 includes a sensor for detecting the position of the transducer array 554 with respect to the housing 552. Using information from the sensor, the processor 116 can determine the angle between any of the planes represented in the images. The processor 116 can also use information from the sensor regarding the position of the transducer array 554 to calculate the position of any portion of the image with respect to the mechanically rotating probe 550. The mechanically rotating probe 550 includes a face (not shown) that is configured to be placed in contact with a patient when acquiring ultrasound data. A clinician may hold the face of the mechanically rotating probe 550 in contact with the patient and obtain images of different planes by rotating the transducer array 554 with respect to the housing 552. According to various embodiments, the processor 116 may cause control the rotation of the transducer array. This allows the clinician to hold the mechanically rotating probe 550 in a fixed position and orientation with respect to both the patient and anatomy being imaged, such as vessel, while acquiring images from different planes. It should be appreciated by those skilled in the art that all the planes acquired with the mechanically rotating probe 550 in a fixed position will intersect each other along the longitudinal axis 108 of the probe.

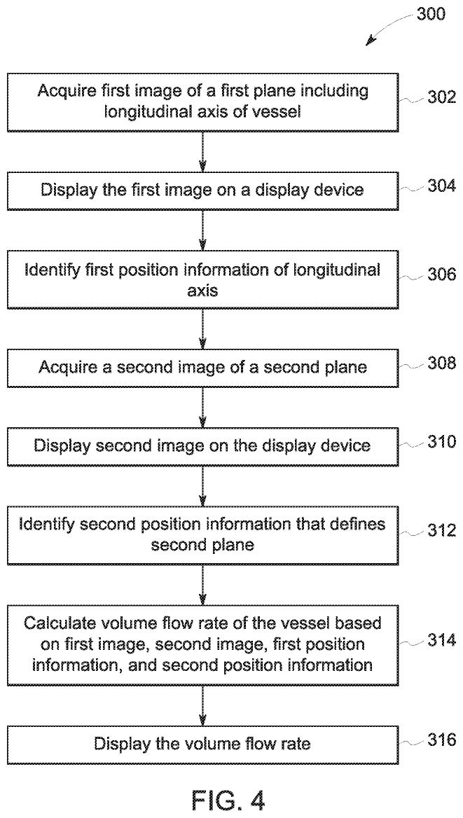

FIG. 4 is a flow chart of a method 300 in accordance with an exemplary embodiment. The individual blocks represent steps that may be performed in accordance with the method 300. Additional embodiments may perform the steps shown in a different sequence, and/or additional embodiments may include additional steps not shown in FIG. 4. The technical effect of the method 300 shown in FIG. 4 is the calculation and display of a volume flow rate based on position information and ultrasound images.

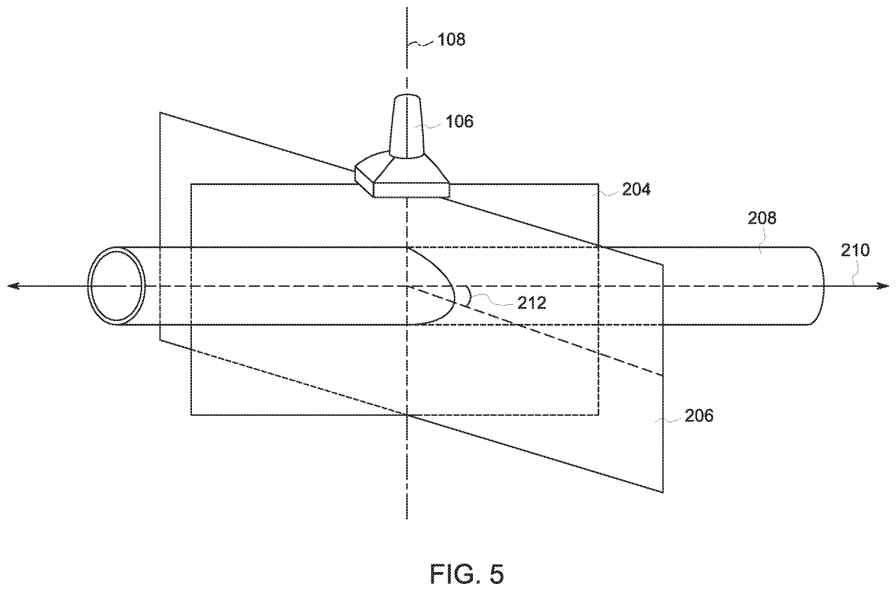

FIG. 5 is a schematic diagram showing the relative orientations of a first plane 204 and a second plane 206 with respect to a vessel 208. The vessel 208 may be an artery or a vein, for example. The vessel 208 includes a longitudinal axis 210. The longitudinal axis 210 is along the centerline of the vessel 208 and may be parallel to the direction blood flows through the vessel according to an embodiment. According to embodiments where the vessel 208 is curved, the longitudinal axis 210 may be parallel to a tangent of a centerline of the vessel 208. The longitudinal axis 210 may be calculated in different ways, or manually identified by a clinician. The ultrasound probe 106 is shown with respect to the first plane 204, the second plane 206, and the vessel 208. As shown in FIG. 5, the first plane 204 includes the longitudinal axis 210 of the vessel 208. For purposes of this disclosure, the phrase “plane includes the longitudinal axis” is defined to mean that the longitudinal axis 210 lies within the first plane 204.

The second plane 206 intersects the longitudinal axis 210 of the vessel 208 at an oblique angle. An angle 212 shown in FIG. 5 represents the angle between the second plane 206 and the longitudinal axis 210 of the vessel 208. FIG. 5 also includes the longitudinal axis 108 of the ultrasound probe 106.

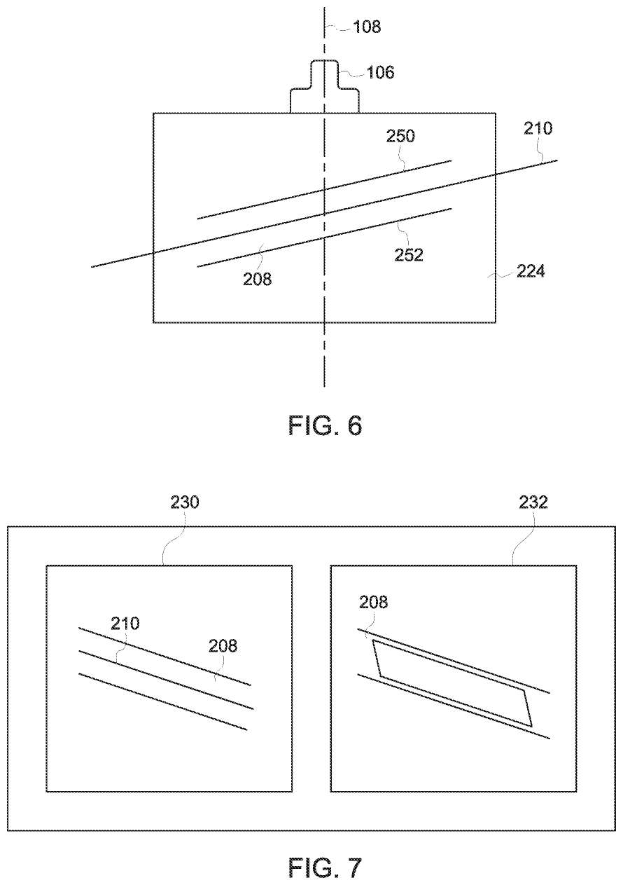

FIG. 6 is a schematic representation of a first image 224 according to an exemplary embodiment. The first image 224 is of the first plane 204 according to an embodiment. FIG. 6 shows the first image 224 with respect to both the ultrasound probe 106 and the longitudinal axis 108 of the ultrasound probe 106. The ultrasound probe 106 and the longitudinal axis 108 of the ultrasound probe 106 show the position of the ultrasound probe 106 during the acquisition of the first image 224.

Referring to the method 300 shown in FIG. 4, at step 302, the processor 116 controls the ultrasound probe 106 to acquire the first image 224 of the first plane 204 with the ultrasound probe 106 in a position with respect to both the patient 210 and the patient's anatomy, such as the vessel. The first plane 204 includes the longitudinal axis 210 of the vessel. The first image 224 may be a static image of a single frame of ultrasound data, or the first image 224 may be a live, or real-time, image sequentially showing a plurality of frames of ultrasound data. Additionally, the first image 224 may include ultrasound data from a single mode or from a plurality of modes. For example, according to an embodiment, the first image 224 may include both B-mode data and colorflow data. The processor 116 may, for instance, control the probe 106 to acquire the colorflow data and the B-mode data in an interleaved manner during step 302.

At step 304, the processor 116 displays the first image 224 on the display device 118. For purposes of this disclosure, the first image 224 may also be referred to as a longitudinal image since the first image 224 includes the longitudinal axis 210 of the vessel 208. As described previously, the first image 224 includes the longitudinal axis 210 of the vessel 208.

According to an embodiment, the processor 116 may control the ultrasound probe 106 to acquire and display multiple images of the first plane 204 at the same time on the display device 118. For example, FIG. 7 is screenshot of an exemplary embodiment where the processor 116 displays two images of the first plane 204 at the same time on the display device. FIG. 7 includes a first B-mode image 230 of the first plane 204 and a first colorflow image 232 of the first plane 204. According to an embodiment, the processor 116 may control the ultrasound probe 106 to acquire colorflow frames of data and B-mode frames of data in an interleaved fashion. For example, the processor 116 may acquire a colorflow frame of data for every N B-mode frames, where N is an interger.

FIG. 7 shows an exemplary embodiment where the processor 116 displays both the first B-mode image 230 of the first plane 204 and the first colorflow image 232 of the first plane 204 on the display device 118 at the same time. Both the first B-mode image 230 and the first colorflow image 232 may be live, or real-time, images that are updated by the processor 116 as additional frames of data are acquired. The first colorflow image 232 may, for instance, be a fusion image of colorflow data and B-mode data. According to other embodiments, the processor 116 may display more than two images of the first plane 204 on the display device 118 at the same time.

At step 306, first position information is identified, where the first position information is the position of the longitudinal axis 210 of the vessel 208 with respect to the ultrasound probe 106. The processor 116 may, for instance, use the location of the longitudinal axis 210 of the vessel in the first image 224 to identify the position of the longitudinal axis 210 of the vessel 208 with respect to the ultrasound probe 106. The processor 116 may use the depth information from the first image 224 and the geometry of the first plane 204 with respect to the probe 106 in order to identify the position of the longitudinal axis 210 of the vessel 208 with respect to the ultrasound probe 106. The position of the longitudinal axis 210 may be determined automatically by the processor 116, semi-automatically with some clinician involvement, or manually by the clinician. According to an embodiment where the position of the longitudinal axis 210 is determined automatically, the processor 116 may use an image processing technique such as edge detection, shape-based object detection, or any other technique in order to determine the position and orientation of the vessel 208. For example, the processor 116 may identify a first edge 250 and a second edge 252 of the vessel 208, as shown in the first image 224, and then, based on the positions of the first edge 250 and the second edge 252, the processor 116 may position the longitudinal axis 210 in the middle of the first edge 250 and the second edge 252. According to an embodiment, a clinician may manually manipulate the position of the ultrasound probe 106 until the ultrasound probe 106 has been positioned so the first image 224 of the first plane 204 includes the longitudinal axis 210 of the vessel. The clinician may, for instance, use feedback from a real-time ultrasound image displayed on the display device 118 in order to correctly position the ultrasound probe 106 so the first image includes the longitudinal axis 210 of the vessel 208.

According to another embodiment, the processor 116 may automatically determine a position for the longitudinal axis 210 based on a colorflow image, such as the first colorflow image 232 shown in FIG. 7. For example, the processor 116 may use the colorflow data to determine the edges of the vessel 208. In some instances where the vessel edges are difficult to determine from B-mode data, the colorflow data may allow for a more accurate determination of the position of the longitudinal axis 210 of the vessel 208. Colorflow data is generated based on Doppler shifts, which is useful for identifying areas of motion in an image. Since the blood is flowing and the vessel edges are relatively stationary, colorflow data may be used to effectively identify the edges of the vessel. Once the edges of the vessel 208 are identified, the processor 116 may automatically or semi-automatically identify the longitudinal axis 210 of the vessel 208. According to another embodiment, the clinician may manually identify the longitudinal axis 210 of the vessel 208 using the first colorflow image 232 for reference. The processor 116 may then determine the position of the longitudinal axis 108 with respect to the ultrasound probe 106 based on the longitudinal axis 210 that was identified.

According to an embodiment where the longitudinal axis 210 is determined semi-automatically, the processor 116 may show an estimated position of the longitudinal axis 210 and may then allow the clinician to manually modify the estimated position of the longitudinal axis 210. The estimated position of the longitudinal axis 210 may be determined based on, for example, any of the methods described hereinabove with respect to the automated techniques.

According to an embodiment, the clinician may manually identify the longitudinal axis 210 on an image of the first plane 204, such as the image 224, the first B-mode image 230, or the first colorflow image 232. For instance, the clinician may use the input device 115 to position a line or other graphic on the longitudinal axis 210 of the vessel on one or more of the first image 224, the first B-mode image 230, and the first colorflow image 232.

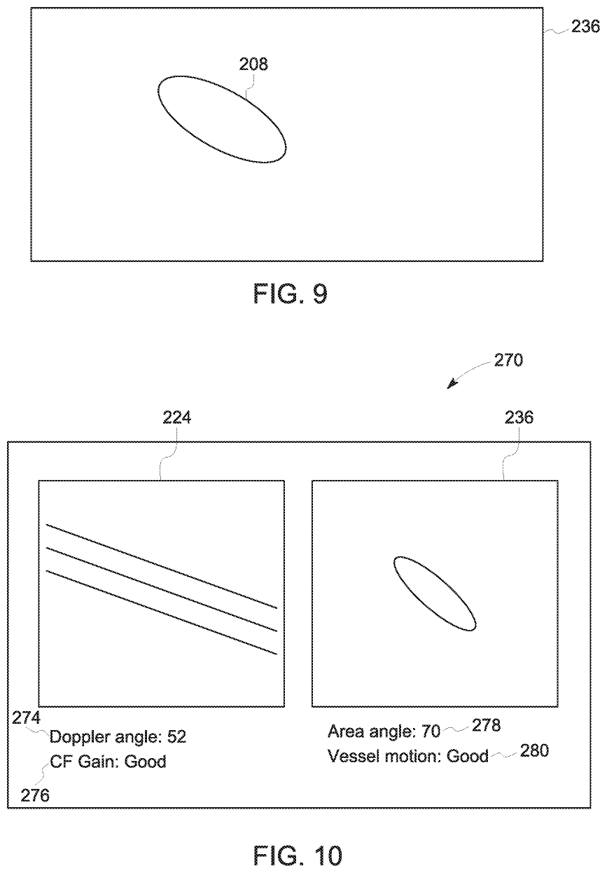

FIG. 9 shows a schematic representation of the second image 236 according to an exemplary embodiment. The second image 236 may be a static image showing a single frame of ultrasound data or the second image 236 may be a live, or real-time, image showing a plurality of frames of data in sequence. The vessel 208 is shown as an ellipse in the second image 236 since the vessel 208 intersects the second plane 206 at an oblique angle. At step 308, the processor 116 controls the ultrasound probe 106 to acquire a second image, such as the second image 236 of the second plane 206. While acquiring the second image, the ultrasound probe 106 remains in the same position with respect to the patient's anatomy being imaged, such as the vessel 208, as the ultrasound probe 106 was in while acquiring the first image 224. In other words, the longitudinal axis 108 of the probe 106 remains in a fixed position with respect to the anatomy being imaged, such as the vessel, while acquiring both the first image 224 and the second image 236. For purposes of this disclosure, the second image 236 may also be referred to as an oblique image since the second plane 206 is at an oblique angle with respect to the longitudinal axis 210. The second image 236 intersects the longitudinal axis 210, and hence the vessel 208, at an oblique angle. In this disclosure, the terms “first,” “second,” and “third,” etc. are used merely as labels, and are not intended to impose numerical requirements or a particular positional order. For example, step 302 may be performed before step 308, or step 302 may be performed after step 308. This means that the first image 224 of the first plane 204 may be acquired before the second image 236 of the second plane 206, or the first image 224 of the first plane 204 may be acquired after the second image 236 of the second plane 206 according to various embodiments. According to an embodiment where the ultrasound probe 106 is an E4D probe, the processor 116 may control the E4D probe to acquire the second image 236 of the second plane 206 by controlling the beamforming of the transducer elements in the E4D probe. According to an embodiment where the ultrasound probe 106 is a mechanically rotating probe, the processor 116 may control a motor in the probe to rotate the transducer array 554 from the position required to acquire the first image 224 of the first plane 204 to the position required to acquire the second image 236 of the second plane 206 while the mechanically rotating probe 550 remains in the same position. In other words, the longitudinal axis 108 of the probe remains in the same position when acquiring both the first image 224 and the second image 236.

At step 310, the second image 236 of the second plane 206 is displayed on the display device 118. At step 312, the processor 116 identifies second position information of the second plane 206 with respect to the probe 106. For embodiments where the ultrasound probe 106 is an E4D probe, such as the E4D probe 500, the processor 116 may identify the second position information based on the position of the second scan plane with respect to the ultrasound probe 106. For embodiments where the ultrasound probe 106 is a mechanically rotating probe, such as the mechanically rotating probe 550, the processor 116 may identify the second position information based on the position of the transducer array 554 with respect to the mechanically rotating probe 550.

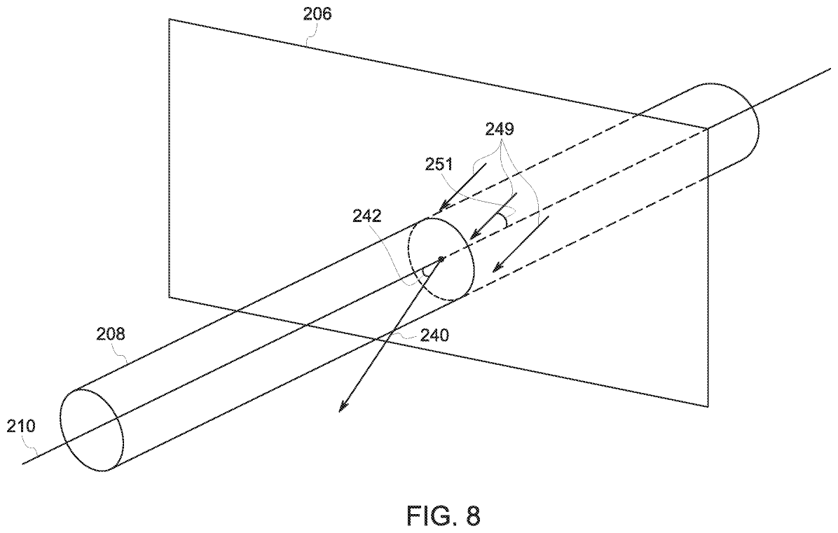

At step 314, the processor 116 calculates a volume flow rate for the vessel 208. According to an embodiment, the processor 116 measures the vessel area from the second image 236 of the second plane 206. The second plane 206 intersects the longitudinal axis 210, and hence the vessel 208, at an oblique angle. This means that the second image 236 includes a sectional view of the vessel 208. FIG. 8 shows the relative positioning of the second plane 206, the vessel 208, and the longitudinal axis 210 of the vessel 208. FIG. 8 also includes a normal vector 240 that is perpendicular, or normal, to the second plane 206. An area angle 242 is defined as the angle between the normal vector 240 and the longitudinal axis 210 of the vessel 208. FIG. 8 also includes a plurality of colorflow beams 249, and a Doppler angle 251 between the colorflow beams 249 and the longitudinal axis 210 of the vessel 208. It should be appreciated based on the description hereinabove that the longitudinal axis 210 is in a different plane than the second plane 206. As such, the Doppler angle 251 represents the angle between the plurality of colorflow beams 249, which may be steered within the second plane 206 and the longitudinal axis 210 of the vessel 208. It is generally desirable to have the Doppler angle 251 be as small as possible in order to have the most accurate velocity measurements within the vessel 208 based on the Doppler data.

At step 314, the processor 116 calculates volume flow rate from the first image 224, the second image 236, the first position information and the second position information. As described hereinabove, the processor 116 may calculate the position of the longitudinal axis 210 with respect to the ultrasound probe 106 based on the first image 224 and the first position information. The processor 116 may use the second image 236 and the second position information to calculate a vessel cross-sectional area. The processor 116 may additionally rely on colorflow data in the second image 236 in combination with the vessel cross-sectional area of vessel 208 to calculate a volume flow rate of the vessel 208. The second image 236 is of the second plane 206. Since the positions of both the longitudinal axis of the vessel 210 and the second plane 206 are known, the processor 116 can calculate the position of the longitudinal axis of the vessel 210 with respect to the second plane 206. The processor 116 may use the relative position of the vessel 210 with respect to the second plane 206 to calculate the vessel cross-sectional area.

According to an embodiment, the processor 116 may determine the vessel cross-sectional area of the vessel 208 based on colorflow data in the second image 236. For example, the colorflow data should show movement only within the vessel 208. According to an exemplary embodiment, the processor 116 may calculate the volume flow rate using Equation 1, shown below:

Volume Flow Rate=Average Velocity*Vessel Cross-Sectional Area Equation 1:

Where Volume Flow Rate is the instantaneous volume flow rate of fluid through a vessel; Average Velocity is the instantaneous spatially-averaged velocity within the vessel's cross section; and Vessel Cross-Sectional Area is the cross-sectional area of the vessel normal to the longitudinal axis.

$$Average Velocity = \frac{{\sum }_{i = 0}^{{N}_{Vessel CF pixels in image 2}} {Vel}_{i} * {α}_{i}}{\begin{matrix}Cos ( Doppler {Angle}_{image 2} ) * \\ {\sum }_{i = 0}^{{N}_{Vessel CF pixels in image 2}} {α}_{i}\end{matrix}} \qquad Equation 2$$

Where N_{Vessel CF pixels in image 2} is the number of colorflow pixels in the second image 236; Velᵢ is the velocity of the ith colorflow pixel; αᵢ is a weighting coefficient for the ith colorflow pixel and Doppler Angle_{image 2} is the angle between colorflow beams and the longitudinal axis 210 of the vessel. The weighting coefficient αᵢ may be set to 1 or may be calculated based on the power of the colorflow at the ith pixel.

Vessel Cross-Sectional Area=Pixels Area_{2nd image}*Cos (Area Angle_{2nd image}) Equation 3:

Where Pixels Area_{2nd image} is the measured area of the colorflow pixels in the second image 224, and the Area Angle_{2nd image} is the angle between the normal vector to the second plane 204 (and the second image 236) and the longitudinal axis 210.

The measured area of the colorflow pixels multiplied by the cosine of the area angle will result in the vessel cross-sectional area. It should be appreciated that other embodiments may use different equations to calculate the volume flow rate based on the first image 224, the second image 236, the first position information, and the second position information. Additionally, according to other embodiments, the processor 116 may either combine some or all of the processing operations described above in one or more different equations, or the processor 116 may separate the processing operations for calculating the volume flow rate into different steps than shown in the above equations. At step 316, the processor 116 displays the volume flow rate on the display device 118.

FIG. 10 is a schematic representation of a screenshot 270 in accordance with an embodiment. According to an exemplary embodiment, the processor 116 may display both the first image 224 and the second image 236 on the display device 118 at the same time. It should be appreciated that only one of the first image 224 (i.e., the longitudinal image) and the second image 236 (i.e., the oblique image) may be live and that the other of the first image 224 and the second image 236 may be either a static frame or a cine loop from a previous acquisition. According to an exemplary embodiment, the first image 224 may be from a previous acquisition and the second image 236 may be a live, or real-time, image.

According to an embodiment, the processor 116 may calculate and display one or more quality parameters on the display device 118. A non-limiting list of quality parameters includes: a Doppler angle 274, a colorflow (CF) gain 276, an area angle 278, and a vessel motion 280. The processor 116 may compare each of the quality parameters to a threshold value to determine whether or not the quality parameter value is within an acceptable range. The processor 116 may use one or more of color, icons, or text to indicate if each of the quality parameters is within an acceptable range. According to an exemplary embodiment, the processor 116 may use color to indicate if each of the quality parameters is within an acceptable range. For example, the processor 116 may display the quality parameter in green if the parameter is within the acceptable range and red if the quality parameter is outside the acceptable range. It should be appreciated that other embodiments may use different colors or a different graphical technique, including text or icons, to indicate if each of the quality parameters is within the acceptable range.

According to an exemplary embodiment, the acceptable range for the Doppler angle may be less than 60 degrees, and the acceptable range for the area angle may be less than 80 degrees. The processor 116 may determine if the colorflow gain is acceptable by calculating a colorflow diameter based on the second, or oblique, image 236 and compare the colorflow diameter to a measured vessel diameter from the B-mode image. Based on this comparison, the processor 116 may calculate if the colorflow image is within the acceptable range for gain. For the vessel motion 280 quality parameter, the processor 116 may detect vessel motion from either the first image 224 or the second image 236 and determine if there is too much vessel motion for a reliable measurement compared to a threshold.

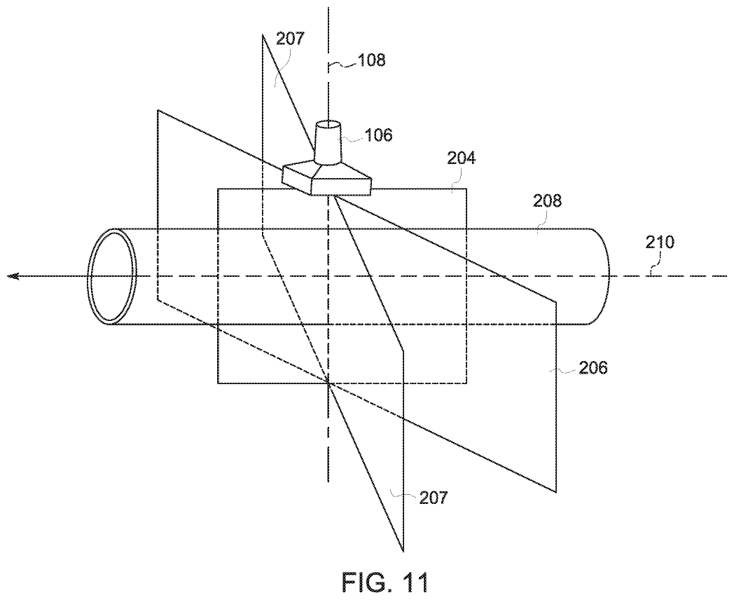

According to another embodiment, images of three different planes of the vessel 208 may be acquired. FIG. 11 is a schematic representation of the first plane 204, the second plane 206, and the third plane 207 in accordance with an embodiment. According to an exemplary embodiment, the first image 224 of the first plane 204 and the second image 236 of the second plane 206 are the same as was previously disclosed hereinabove. The first plane 204 includes the longitudinal axis 210 of the vessel 208, and the second plane 206 is oblique to the longitudinal axis 210. For example, in addition to the first, or longitudinal, image 224 of the first plane 204 and the second, or oblique, image 236 of the second plane 206, the clinician may also use the probe 106 to acquire a third, or transverse, image 287 of a third plane 207. The third plane 207 is transverse to the longitudinal axis 210 of the vessel 208.

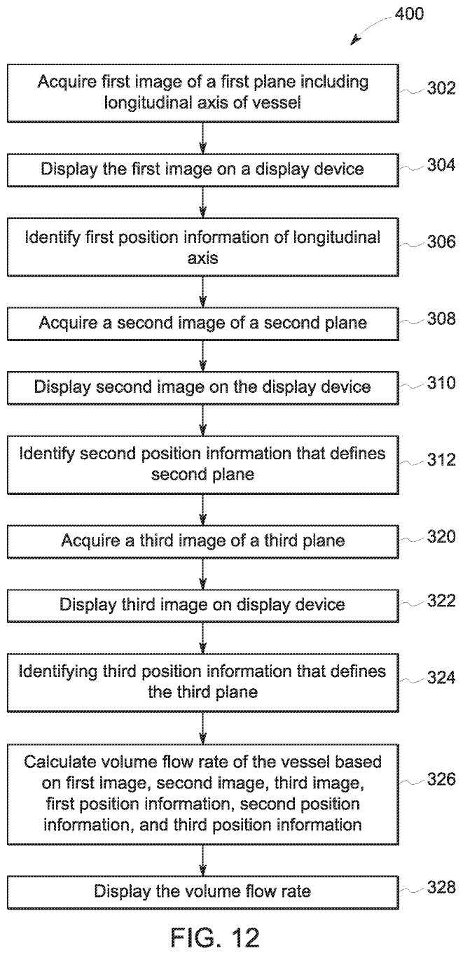

FIG. 12 is a flow chart of a method 400 in accordance with an exemplary embodiment. The individual blocks represent steps that may be performed in accordance with the method 400. Additional embodiments may perform the steps shown in a different sequence, and/or additional embodiments may include additional steps not shown in FIG. 12. The technical effect of the method 400 shown in FIG. 12 is the calculation and display of a volume flow rate based on position information and ultrasound images.

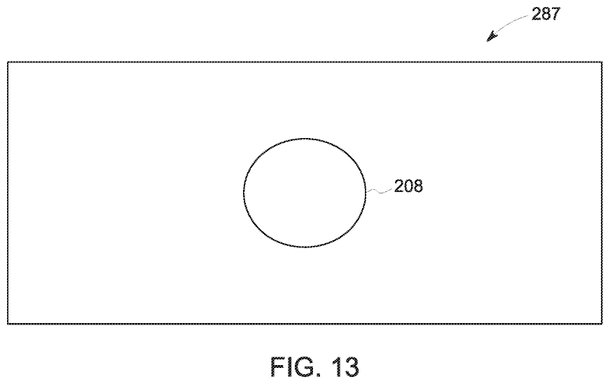

Steps 302, 304, 306, 308, 310, and 312 of the method 400 were previously described with respect to the method 300, and therefore, they will not be described again. FIG. 13 is a third image 287 of the third plane 207 in accordance with an embodiment. At step 320, the clinician acquires a third image of a third plane, such as the third image 287 of the third plane 207. The third plane 207 is transverse to the longitudinal axis 210 of the vessel 208 and the longitudinal axis 108 of the probe 106 may be in the same orientation during the acquisition of the first image 224, the second image 236, and the third image 287. Since the positions of the longitudinal axis 210 of the vessel, the first plane 204, the second plane 206, and the third plane 207 are all known with respect to the probe 106, the processor 116 may calculate the relative positions and geometries between the first plane 204, the second plane 206, the third plane 207, and the longitudinal axis 210 of the vessel. The clinician does not need to move the ultrasound probe 106 to a different position or to tilt the ultrasound probe 106 to acquire the first image 224, the second image 236, or the third image 287. The first image 224 of the first plane 204, the second image 236 of the second plane 206, and the third image 287 of the third plane 207 may be acquired in any order according to various embodiments.

The third plane 207 is transverse to the vessel 208. According to an embodiment, the processor 116 may calculate the vessel diameter from the third, or transverse, image 287. Since the third plane 207 is transverse to longitudinal axis 210 of the vessel 208, it may not be necessary to apply a cosine adjustment to the measured area of the vessel from the third image 287. Those skilled in the art will appreciate that the cross-section of the vessel 208 will be less elliptical in the third image 287 because the third plane 207 is transverse to the longitudinal axis 210 of the vessel 108. If the longitudinal axis 210 is perpendicular to the third plane 207, then it is not necessary to apply a cosine adjustment to the measured area of the vessel 208. If, however, the longitudinal axis 210 is not exactly perpendicular to the third plane 207, such as when the longitudinal axis 210 is not parallel to the skin of the patient, it will still be necessary to apply a cosine adjustment to the measure area of the vessel 208 from the third image 287. However, for most circumstances, determining the area of the vessel from the third, or transverse, image 287, will result in a smaller cosine adjustment compared to calculating the area from the second, or oblique, image 236 as described with respect to the method 300. Applying a smaller cosine adjustment to the area measurement should result in a more accurate calculation for the area of the vessel. In other embodiments, the third plane 207 may be perpendicular to the longitudinal axis 210.

At step 322, the processor 116 displays the third image 287 on the display device 118. The third image 287 may be displayed with one or both of the first image 224 and the second image 236, or the third image 287 may be displayed without any other ultrasound images.

At step 324, the processor 116 identifies third position data of the third plane 107 with respect to the ultrasound probe 106. For embodiments where the ultrasound probe 106 is an E4D probe, such as the E4D probe 500, the processor 116 may identify the third position information based on the position of the third scan plane with respect to the ultrasound probe 500. For embodiments where the ultrasound probe 106 is a mechanically rotating probe, such as the mechanically rotating probe 550, the processor 116 may identify the third position information based on the position of the transducer array 554 with respect to the mechanically rotating probe 550.

At step 326, the processor uses the first image 224, the second image 236, the third image 287, the first position information, the second position information, and the third position information to calculate the volume flow rate of the vessel 208. The following equations (Equation 4, Equation 5, and Equation 6) may be used to calculate the volume flow rate:

Volume Flow Rate=Average Velocity*Vessel Cross-Sectional Area Equation 4:

Where Volume Flow Rate is the instantaneous volume flow rate of fluid through a vessel; Average Velocity the instantaneous spatially-averaged velocity within the vessel's cross section; and Vessel Cross-Sectional Area is the cross-sectional area of the vessel normal to the longitudinal axis.

$$Average Velocity = \frac{{\sum }_{i = 0}^{{N}_{Vessel CF pixels in image 2}} {Vel}_{i} * {α}_{i}}{\begin{matrix}Cos ( Doppler {Angle}_{image 2} ) * \\ {\sum }_{i = 0}^{{N}_{Vessel CF pixels in image 2}} {α}_{i}\end{matrix}} \qquad Equation 5$$

Where N_{Vessel CF pixels in image 2} is the number of colorflow pixels in the second image 224; Velᵢ is the velocity of the ith colorflow pixel; αᵢ is a weighting coefficient for the ith colorflow pixel and Doppler Angle _{image 2} is the angle between colorflow beams and the longitudinal axis 210 of the vessel. The weighting coefficient αᵢ maybe set to 1 or maybe calculated based on the power of the colorflow at the ith pixel.

Vessel Cross Sectional Area=Pixels Area_{image 3}*Cos(Area Angle _{Image 3}) Equation 6:

Where Pixels Area_{Image 3} is the measured area of the vessel's pixels in the third image 287, and the Area Angle_{Image 3} is the angle between the normal vector to the third plane 207 (and the third image 287) and the longitudinal axis 210.

It should be appreciated that other embodiments may use different equations to calculate the volume flow rate based on the first image 224, the second image 236, the third image 287, the first position information, the second position information, and the third position information. Additionally, according to other embodiments, the processor 116 may separate the processing operations for calculating the volume flow rate into a plurality of separate steps. According to an embodiment using the third image 287 of the third plane 207, the area angle is defined to be the angle between a normal vector to the third plane 207 and the longitudinal axis 210 of the vessel 208, and the pixel area would be calculated from the third, or transverse, image 287. The vessel CF pixels, on the other hand, would be determined from the second, or oblique, image 236. According to an embodiment, the processor 116 may be configured to use the first position information, the second position information, and the third position information to calculate the position of the longitudinal axis 210 and the first plane 204, the second plane 206, and the third plane 207 with respect to a 3D coordinate system. Next, at step 328, the processor 116 displays the volume flow rate on the display device 118.

Both the method 300 and the method 400 have numerous advantages over conventional methods. As described hereinabove, it is generally desirable to have as low a Doppler angle as possible in order to obtain the most accurate and reliable flow velocity measurements. Conventional methods typically involve tiling the ultrasound probe 106 in order to reduce the Doppler angle. However, there is a limit to how far the ultrasound probe 106 can be tipped before the ultrasound probe 106 is no longer in good contact with the patient's skin for the transmission and reception of ultrasound energy. By using a technique where the longitudinal axis 108 of the probe 106 remains in the same position while acquire images of multiple different planes, the elements 104 of the ultrasound probe 106 remain in good acoustic contact with the patient while acquiring the colorflow data. This allows the clinician to select a second position that is optimized for acquiring colorflow data without being limited by poor acoustic contact. As a contrast, conventional techniques suffer from poor acoustic contact at tilt angles where the longitudinal axis 108 of the probe is greater than 20 degrees from normal to the patient's skin. Various embodiments of this invention allow for a lower Doppler angle compared to conventional techniques, which allows for the acquisition of more accurate colorflow data.

Additionally, even lower Doppler angles can be achieved with embodiments of the present invention because it is possible to apply steering to the colorflow beams transmitted within the second plane 206 to acquire the colorflow data. Depending upon the orientation of the vessel, steering the colorflow beams may lead to smaller Doppler angles, and thus significantly more accurate velocity measurements. For conventional techniques relying on tilting the probe, in-plane beam steering is transverse to the longitudinal axis 210 of the vessel 208, so steering angle does not result in similar improvement in Doppler angles for the acquisition of colorflow data.

The technique used in method 300 and method 400 results in a more accurate area measurement because the vessel area is based on a measured vessel area in either the second image 236 (i.e., the oblique image) or the third image 287 (i.e., the transverse image). This overcomes a limitation of conventional techniques where the cross-section of the vessel is assumed to be circular. Assuming that the vessel is circular may lead to significant inaccuracies for embodiments where the vessel cross-section is far from circular. Embodiment of the invention are more accurate than conventional techniques because the vessel cross-sectional area is measured from ultrasound images rather than assuming a circular cross-section for cross-sectional area calculations.

As discussed in the background, conventional techniques typically use pulsed wave (PW) Doppler acquired from a relatively small range gate, and the assumption that the velocity derived from within the range gate can be applied to the whole cross-sectional area of the vessel 208. For situations where the velocity within the vessel varies, the conventional technique of extrapolating and/or applying the measured velocity within the range gate to the whole vessel can also be a significant source of error. In contrast, by basing the velocity on colorflow data acquired for the whole cross-section of the vessel 208, embodiments of the invention provide much more accurate flow velocities across the whole vessel cross-section, which in turn leads to greater levels of accuracy for calculating a volume flow rate for the vessel.

Embodiments of the present inventions may also be configured to provide real-time volume flow rates to the clinician as the clinician is performing the ultrasound scan. These embodiments are more accurate than conventional techniques for the reasons discussed hereinabove. Embodiments of the present invention therefore provide reliable techniques for calculating volume flow rates in real-time with a much great accuracy than conventional techniques. Providing the clinician with real-time volume flow rates allows the clinician to monitor volume flow-rates of patients more closely, which may be advantageous for some clinical situations where a change in the volume flow-rate could provide the clinician with an early warning of a potentially problematic clinical scenario.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. Moreover, in the following claims, the terms “first,” “second,” and “third,” etc. are used merely as labels, and are not intended to impose numerical requirements on their objects.

What is claimed is:

1. A method for calculating a volume flow rate using ultrasound, the method comprising:
acquiring, with an ultrasound probe, a first image of a first plane, where the first plane includes a longitudinal axis of a vessel;
displaying the first image on a display device;
identifying, with a processor, first position information, where the first position information is of the longitudinal axis with respect to the ultrasound probe;
acquiring, with the ultrasound probe, a second image of a second plane that intersects the longitudinal axis of the vessel at an oblique angle, where the second plane is rotated about a longitudinal axis of the ultrasound probe with respect to the first plane, where the ultrasound probe is in the same position with respect to the vessel when acquiring both the first image of the first plane and the second image of the second plane;
displaying the second image on the display device;
identifying, with the processor, second position information, where the second position information defines the second plane with respect to the ultrasound probe;
calculating, with the processor, a volume flow rate of the vessel based on the first image, the second image, the first position information, and the second position information; and
displaying the volume flow rate on a display device.

2. The method of claim 1, wherein the ultrasound probe is an E4D ultrasound probe.

3. The method of claim 1, wherein the ultrasound probe is a mechanically rotating probe.

4. The method of claim 1, wherein calculating the volume flow rate comprises identifying a contour of the vessel in the second image and using the contour to calculate a vessel cross-sectional area.

5. The method of claim 4, wherein the second image comprises B-mode data, and wherein identifying the contour of the vessel comprises identifying the contour based on the B-mode data in the second image.

6. The method of claim 4, wherein the second image comprises colorflow data, and wherein identifying the contour of the vessel comprises identifying the contour based on the colorflow data in the second image.

7. The method of claim 4, wherein acquiring the second image comprises acquiring colorflow data along a plurality of colorflow beams, and wherein calculating the volume flow rate further comprises using the first position information and the second position information to calculate a Doppler angle between the plurality of colorflow beams and the longitudinal axis of the vessel.

8. The method of claim 1, further comprising:
acquiring a third image of a third plane intersecting the vessel, where the third plane is transverse to the longitudinal axis of the vessel, where the ultrasound probe is in the same position with respect to the vessel when acquiring the third image of the third plane, the first image of the first plane, and the second image of the second plane;
identifying, with the processor, third position information, where the third position information defines the third plane with respect to the ultrasound probe;
displaying the third image on the display device; and
wherein calculating the volume flow rate is also based on the third image and the third position information.

9. The method of claim 8, wherein calculating the volume flow rate comprises identifying a contour of the vessel in the third image and calculating an area of the vessel based on the contour.

10. The method of claim 8, wherein calculating the volume flow rate further comprises calculating a vessel cross-sectional area based on the third position information and the first position information.

11. The method of claim 10, wherein acquiring the second image comprises acquiring colorflow data along a plurality of colorflow beams, and wherein calculating the volume flow rate further comprises using the first position information and the second position information to calculate a Doppler angle between the plurality of colorflow beams and the longitudinal axis of the vessel.

12. The method of claim 1, wherein calculating the volume flow rate is performed in real-time.

13. An ultrasound imaging system comprising:
an ultrasound probe comprising a plurality of elements;
a display device;
a processor in electronic communication with the ultrasound probe and the display device, wherein the processor is configured to:
control the ultrasound probe to acquire a first image of a first plane, wherein the first plane is positioned to include a longitudinal axis of a vessel;
display the first image on the display device;
identify first position information of the longitudinal axis of the vessel with respect to the ultrasound probe;
control the ultrasound probe to acquire a second image of a second plane, wherein the second plane is rotated about a longitudinal axis of the ultrasound probe from the first plane, and wherein the ultrasound probe is in the same position with respect to the vessel when acquiring both the first image of the first plane and the second image of the second plane;
display the second image on the display device;
identify second position information, where the second position information defines the second plane with respect to the ultrasound probe;
calculate a volume flow rate of the vessel based on the first image, the second image, the first position information and the second position information; and
display the volume flow rate on the display device.

14. The ultrasound imaging system of claim 13, wherein the ultrasound probe is an E4D probe.

15. The ultrasound imaging system of claim 13, wherein the ultrasound probe is a mechanically rotating probe.

16. The ultrasound imaging system of claim 13, wherein the processor is further configured to automatically identify a contour of the vessel in the second image and use the contour of the vessel to calculate vessel cross-sectional area.

17. The ultrasound imaging system of claim 13, wherein the processor is further configured to:
control the ultrasound probe to acquire a third image of a third plane, wherein the third plane is transverse to the longitudinal axis of the vessel, where the ultrasound probe is in the same position with respect to the vessel while acquiring the third image of the third plane, the first image of the first plane, and the second image of the second plane.

18. The ultrasound imaging system of claim 17, wherein the processor is further configured to calculate the volume flow rate by using the third image to calculate an area of the vessel.

19. The ultrasound imaging system of claim 13, wherein the processor is configured to display the volume flow rate of the vessel in real-time.

20. The ultrasound imaging system of claim 13, wherein the second image includes colorflow data.

21. The ultrasound imaging system of claim 13, wherein the second image include B-mode data.
